(19) United States
(12) Patent Application Publication
(10) Pub. No.: US 2023/0034709 A1
(43) Pub. Date: Feb. 2, 2023

(54) METHOD AND APPARATUS FOR ANALYING EXPERIENCED DIFFICULTY

(71) Applicant: DAEKYO CO., Ltd., Seoul (KR)

(72) Inventor: Ho Jun KANG, Seoul (KR)

(21) Appl. No.: 17/870,777

(22) Filed: Jul. 21, 2022

(30) Foreign Application Priority Data
Jul. 23, 2021 (KR) 10-2021-0096999

(51) Int. Cl. A61B 5/16; G09B 19/00; G06V 40/16; G06T 7/246; G06T 7/90; A61B 5/024

(52) U.S. Cl. CPC A61B 5/165; G09B 19/00; G06V 40/172; G06T 7/246; G06T 7/90; A61B 5/024; G09B 7/02

(57) ABSTRACT

The present disclosure provides an experienced difficulty analysis method and apparatus. According to one embodiment of the present disclosure, the experienced difficulty analysis method and apparatus are provided for acquiring biometric information of a learner to obtain micro-vibration information of face region of the learner, and determining experienced difficulty of the learner for learning content by using the micro-vibration information.

### CROSS-REFERENCE TO RELATED APPLICATIONS

This application is based on, and claims priority from, Korean Patent Application Number 10-2021-0096999, filed on Jul. 23, 2021, the disclosure of which is incorporated by reference herein in its entirety.

### BACKGROUND

### Technical Field

The present disclosure relates to a method and apparatus for analyzing experienced difficulty.

### Discussion

The contents described in this section simply provide background information on the present disclosure and do not constitute the prior art.

Unlike learning difficulty of learning contents, experienced difficulty is flexible since it may vary depending on emotional or physical state of a learner. When a learner learns learning content with an excessively high or low level of experienced difficulty, the learner's learning efficiency decreases. Accordingly, the learning content provider need to grasp the learner's experienced difficulty accurately and provide learning content based on the learner's experienced difficulty.

However, the current experienced difficulty is identified by conducting a survey on a sample of learners. Accordingly, it is difficult to ensure the objectivity of the determination result of the experienced difficulty, and it takes a long time to obtain the determination result. As a result, it is difficult for the learning content provider to select and provide appropriate learning content in consideration of the learner's experienced difficulty.

### SUMMARY

In view of the above, the present disclosure provides a method and apparatus for analyzing experienced difficulty, capable of obtaining micro-vibration information of a face region of a learner by acquiring biometric information of the learner, and determining the learner's experienced difficulty for learning content using the micro-vibration information.

Further, the present disclosure provides a method and apparatus for analyzing experienced difficulty, capable of providing learning content based on determined experienced difficulty.

The aspects to be achieved by the present disclosure are not limited to the above-mentioned objects, and other objects not mentioned may be clearly understood by those skilled in the art from the following description.

According to one embodiment of the present disclosure, an experienced difficulty analysis method comprising acquiring image information including a face region of a learner who learns learning content as biometric information of the learner; obtaining micro-vibration information of the face region from the biometric information; and determining experienced difficulty of the learner with respect to the learning content using the micro-vibration information is provided.

According to one embodiment of the present disclosure, in the above-described experienced difficulty analysis method, the experienced difficulty analysis method further comprising updating a method of providing learning content based on the experienced difficulty is provided

According to one embodiment of the present disclosure, a computer program stored in one or more non-transitory computer-readable recording medium to execute the experienced difficulty analysis method according to any one of the experienced difficulty analysis method described above is provided.

According to one embodiment of the present disclosure, An experienced difficulty analysis apparatus comprising a biometric information acquisition unit configured to acquire image information including a face region of a learner as biometric information of the learner; a biometric information analyzer configured to obtain micro-vibration information of the face region from the biometric information; and an experienced difficulty determination unit configured to determine experienced difficulty of the learner with respect to learning content which the learner learns using the micro-vibration information is provided.

According to one embodiment of the present disclosure, by acquiring biometric information of the learner to obtain micro-vibration information of the learner's face region, and determining the learner's experienced difficulty for learning content using the micro-vibration information, it is possible to accurately determine the experienced difficulty based on the biometric information of the learner, and the determination of the experienced difficulty can be automatically performed.

According to one embodiment of the present disclosure, as the content providing method is updated according to the experienced difficulty, it is possible to provide learning content customized for a learner in consideration of the learner's experienced difficulty.

Effects of the present disclosure are not limited to the above-mentioned effects, and other effects not mentioned will be clearly understood by those skilled in the art from the following description.

### BRIEF DESCRIPTION OF THE DRAWINGS

FIG. 1 is a block diagram illustrating an experienced difficulty analysis apparatus according to one embodiment of the present disclosure.

FIG. 2 is a flowchart illustrating an experienced difficulty analysis method according to one embodiment of the present disclosure.

### DETAILED DESCRIPTION

Hereinafter, some exemplary embodiments of the present disclosure will be described in detail with reference to the accompanying drawings. In the following description, like reference numerals preferably designate like elements, although the elements are shown in different drawings. Further, in the following description of some embodiments, a detailed description of known functions and configurations incorporated therein will be omitted for the purpose of clarity and for brevity.

Additionally, various terms such as first, second, A, B, (a), (b), etc., are used solely for the purpose of differentiating one component from others but not to imply or suggest the substances, the order or sequence of the components. Throughout this specification, when parts “include” or “comprise” a component, they are meant to further include other components, not excluding thereof unless there is a particular description contrary thereto. The terms such as “unit,” “module,” and the like refer to units for processing at least one function or operation, which may be implemented by hardware, software, or a combination thereof.

The detailed description to be disclosed below in conjunction with the accompanying drawings is intended to illustrate exemplary embodiments of this disclosure and is not intended to illustrate the only embodiments that this disclosure may be implemented.

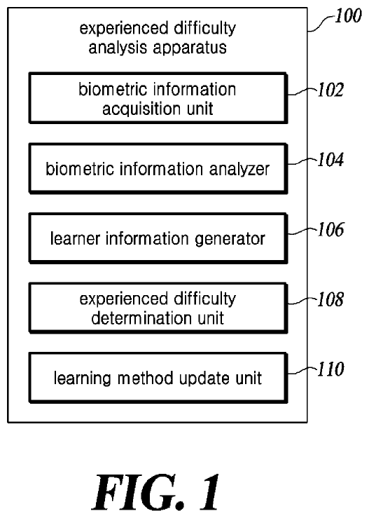

FIG. 1 is a block diagram illustrating an experienced difficulty analysis apparatus according to one embodiment of the present disclosure.

The experienced difficulty analysis apparatus 100 according to one embodiment of the present disclosure includes all or part of a biometric information acquisition unit 102, a biometric information analyzer 104, a learner information generator 106, an experienced difficulty determination unit 108, and a learning method update unit 110. The experienced difficulty analysis apparatus 100 shown in FIG. 1 is exemplified as an embodiment of the present disclosure, and not all components shown in FIG. 1 are essential components, and some components may be added, changed or deleted in another embodiment. For example, in another embodiment, the experienced difficulty analysis apparatus may further include a database (not shown) for storing learning content, and in still another embodiment, the experienced difficulty analysis apparatus may not include the learning method update unit and may be configured to transmit determined experienced difficulty information to a content provider (not shown) that determines and provides learning content.

FIG. 1 illustrates the experienced difficulty analysis apparatus 100 as a apparatus, but this is for convenience of description, and in another embodiment, the experienced difficulty analysis apparatus may be implemented as a software module or a processor that performs the functions of the components 102 to 110.

The biometric information acquisition unit 102 acquires biometric information of a learner. Here, the biometric information is image information including a face region of the learner. Such image information may be, for example, information composed of a plurality of frames per second. In addition, in order for the experienced difficulty analysis apparatus 100 to determine experienced difficulty of the learner with respect to each of learning contents provided to the learner, the image information obtained by the biometric information acquisition unit 102 may be image information obtained during the learning time of the learning content. In this case, the experienced difficulty analysis apparatus 100 may analyze changes in the learner's face region (e.g., occurrence of vibration, skin color change, etc.) from the time when learning of the learning content starts to the time when the learning ends, so that more accurate experienced difficulty analysis is possible. Alternatively, the image information may be configured to include 15 to 60 frames per second, so that the learner's experienced difficulty analysis can be performed accurately while being performed within an appropriate execution time. The data size, resolution, or the number of frames per second of the image information acquired by the biometric information acquisition unit 102 may vary depending on the computing resources available to the experienced difficulty analysis apparatus 100.

The biometric information acquisition unit 102 may obtain biometric information from a capture apparatus (not shown) mounted to the experienced difficulty analysis apparatus 100, or obtain biometric information by obtaining biometric information from a learner apparatus (not shown) or a server (not shown). The biometric information acquisition unit 102 may pre-process the acquired biometric information. For example, the biometric information acquisition unit 102 may decode the acquired image information, crop the size of the image information to the size of computable data, or synchronize the acquired image information, but is not limited thereto.

The biometric information analyzer 104 analyzes the biometric information to obtain micro-vibration information and/or fine skin color change amount of the learner's face region. The biometric information analyzer 104 may extract position information of the face region from the image information to obtain micro-vibration information. The face position information may include upper left coordinate information of the face region and width information and height information of the face region, but this is only an example.

The biometric information analyzer 104 may obtain micro-vibration information of the face region by measuring a difference between position information of the face region for frames of the image information that is biometric information.

As another example, the biometric information analyzer 104 may extract position information of the face region from the biometric information by using a pre-learned face classification model, and perform face modeling based on the position information of the face region to recognize feature points on the face region and/or texture information of the face region. In this case, the pre-learned face classification model may be a model in which machine learning-based or deep learning-based learning is performed to receive an image and classify a face region. Thereafter, the biometric information analyzer 104 may obtain micro-vibration information by calculating a change amount of the feature point or a change amount of the texture information.

The biometric information analyzer 104 may obtain micro-vibration information due to vestibular-emotional reflex (VER) from the biometric information. Human emotions are expressed as micro-movements (or micro-vibrations) of humans (or human heads) through the human vestibular system, and these movements are called vestibular-emotional reflexes. The vestibular system controls the human senses of position and balance, and functions to maintain the human head in a vertical equilibrium state. When human emotions are unstable, that is, when there is a change in emotions, an imbalance occurs in the body's sense of balance, and the vestibular system functions to correct this imbalance. In this process, micro-movements (or micro-vibrations) occur in the body. When emotional information or physical state information is calculated based on the vestibular-emotional reflex, the emotional information or physical state information can be inferred using image information, which is non-contact information. The biometric information analyzer 104 may obtain micro-vibration information due to vestibular-emotional reflex from the biometric information by using a vestibular-emotional reflex detection algorithm which a person skilled in the art may employ.

The biometric information analyzer 104 may measure the amount of change in skin color of the learner (or the learner's face region) recognized from image information that is biometric information.

The learner information generator 106 generates learner information using the micro-vibration information and/or the fine skin color change amount. In this case, the learner information is information used to determine the experienced difficulty, which is information about the learner calculated based on the biometric information. The learner information may be, for example, emotional information and/or physical state information of the learner. Alternatively, the learner information may be the learner's valence and arousal.

Specifically, the learner information generator 106 may calculate the learner's emotional information and/or the learner's physical state information using the micro-vibration information and/or the fine skin color change amount. The emotional information may be, for example, information about at least one of emotion classifications such as joy, surprise, sadness, anger, interest, stress, fear, boredom, pleasantness, displeasure, nervousness, frustration, and neutral. Such emotional information may be information of a main emotion classification to which the learner belongs, information of a plurality of emotion classifications to which the learner belongs, or weight information of a plurality of emotion classifications to which the learner belongs, but is not limited thereto.

The learner information generator 106 may calculate the learner's heart rate from the fine skin color change amount. Since the change in skin color is due to a change in blood volume under the skin epidermis, the heart rate (or the change in heart rate, which will be omitted hereinafter) may be calculated by estimating the change in blood volume from the fine skin color change amount. The learner information generator 106 may calculate the learner's heart rate from the fine skin color change (or the change in blood volume) by using remote photoplethysmography (RPPG) technology. The RPPG technology is a technology that measures the reflected light from human skin and measures the heart rate based on the degree of change in the reflected light. This technology uses the point that the amount of light absorbed by the human body and the amount of scattered light change depending on the blood volume in the blood vessel.

The learner information generator 106 may calculate the learner's emotional information and/or the learner's physical state information using the micro-vibration information and the learner's heart rate. The learner information generator 106 may calculate the learner's emotional information and/or the learner's physical state information using an algorithm that maps micro-vibration information and heart rate to human electroencephalogram (EEG) information as parameters.

The experienced difficulty determination unit 108 determines the learner's experienced difficulty with respect to the learning content using the micro-vibration information and/or the fine skin color change amount information. Specifically, the experienced difficulty determination unit 108 may determine the learner's experienced difficulty based on emotional information and/or physical state information calculated using micro-vibration information and/or fine skin color change information (or heart rate). The experienced difficulty determination unit 108 may determine the experienced difficulty using an algorithm for determining the experienced difficulty using emotional information and/or physical state information as parameters or a pre-learned experienced difficulty determination model.

The experienced difficulty determination unit 108 may determine the experienced difficulty by calculating valence and arousal of the learner based on the emotional information and/or the physical state information. The valence and arousal are numerical values indicating the strength of an emotional stimulus. The valence is a number that evaluates whether a corresponding stimulus induces positive or negative emotions, and the arousal refers to the emotional strength of the corresponding stimulus. The specific method of calculating the valence and arousal depends on a means that a person skilled in the art can employ.

The experienced difficulty determination unit 108 may determine the learner's experienced difficulty level by using a pre-set experienced difficulty mapping table for the valence and arousal. An example of the experienced difficulty mapping table is shown in Table 1.

TABLE 1

| | Arousal | |
|---|---|---|
| Valence | Positive | Negative |
| Positive | Low experienced difficulty | Slightly low experienced |
| | | difficulty |
| Negative | Slightly high experienced | High experienced difficulty |
| | difficulty | |

The learning method update unit 110 updates a method of providing learning content based on the determined experienced difficulty. For example, the learning method update unit 110 may select learning content having learning difficulty slightly higher than the experienced difficulty in order to enhance the learner's achievement. Or, in order to arouse the interest of the learner, learning content having learning difficulty slightly lower than the experienced difficulty may be selected. Or, in order to repeatedly train the learner, learning content having a level of learning difficulty similar to that of the experienced difficulty may be selected. Such selection may be performed using non-biological information about the learner.

Non-biometric information is information other than biometric information that is collected while the learner performs learning and, for example, may include all or part of a time when learning content is provided, information input by the learner (e.g., answers to questions, handwriting, voice, etc.), a time required for the input of the learner (e.g., time to solve questions, time to write answers), a time when the learner starts to learn learning content, whether the learner has used supplemental content (e.g., expert commentary, online lectures, etc.), but is not limited thereto. The non-biometric information may be information generated and stored in such a way that an identifier of the learning content or an identifier of a bundle to which the learning content belongs is recorded according to an action type of the learner's behaviors. Such non-biometric information may be acquired from an input unit (not shown) mounted in the experienced difficulty analysis apparatus 100 or by receiving the non-biometric information from a learner apparatus (not shown) or a database (not shown), but the present disclosure is not limited to thereto.

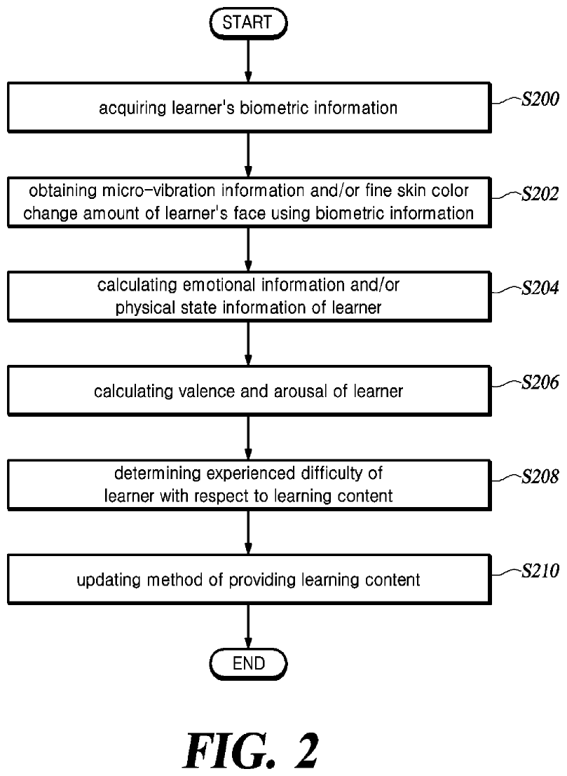

FIG. 2 is a flowchart illustrating an experienced difficulty analysis method according to one embodiment of the present disclosure.

The experienced difficulty analysis apparatus acquires biometric information of a learner (S200). The experienced difficulty analysis apparatus may acquire image information including a face region of the learner who learns learning content as biometric information.

The experienced difficulty analysis apparatus obtains micro-vibration information of the learner's face region (or head) and/or an amount of fine skin color change in the learner's face region using the biometric information (S202). Here, the micro-vibration information may be micro-vibration information due to the vestibular-emotional reflex.

The experienced difficulty analysis apparatus calculates emotional information and/or physical state information of the learner using the micro-vibration information and/or the fine skin color change amount (S204). In another embodiment, step S204 may be omitted.

The experienced difficulty analysis apparatus calculates valence and arousal of the learner based on the learner's emotional information and/or physical state information (S206). In another embodiment, step S206 may be omitted.

The experienced difficulty analysis apparatus determines experienced difficulty of the learner with respect to the learning content using the micro-vibration information and/or the fine skin color change (S208). In another embodiment, the experienced difficulty analysis apparatus may determine experienced difficulty using the learner's emotional information and/or physical state information calculated in step S204, or using the valence and arousal calculated in step S206.

The experienced difficulty analysis apparatus updates the method of providing learning content to the learner based on the determined experienced difficulty (S210). For example, the learning difficulty, the learning quantity, and the type of learning content to be provided to the learner may be adjusted based on the determined experienced difficulty, but the present disclosure is not limited thereto.

Although FIG. 2 presents the respective steps thereof as being sequentially performed, it merely instantiates the technical idea of some embodiments of the present disclosure. Therefore, a person having ordinary skill in the pertinent art could incorporate various modifications, additions, and substitutions in practicing the present disclosure by changing the sequence of steps illustrated by FIG. 2 or by performing one or more of the steps thereof in parallel, and hence the steps in FIG. 2 are not limited to the illustrated chronological sequences.

Various implementations of the systems and methods described herein may be realized by digital electronic circuitry, integrated circuits, field-programmable gate arrays (FPGAs), application-specific integrated circuits (ASICs), computer hardware, firmware, software, and/or their combination. These various implementations can include those realized in one or more computer programs executable on a programmable system. The programmable system includes at least one programmable processor coupled to receive and transmit data and instructions from and to a storage system, at least one input apparatus, and at least one output apparatus, wherein the programmable processor may be a special-purpose processor or a general-purpose processor. Computer programs, which are also known as programs, software, software applications, or codes, contain instructions for a programmable processor and are stored in a “computer-readable recording medium.”

The computer-readable recording medium includes any types of recording apparatus on which data that can be read by a computer system are recordable. Examples of computer-readable recording medium include non-volatile or non-transitory media such as a ROM, CD-ROM, magnetic tape, floppy disk, memory card, hard disk, optical/magnetic disk, storage apparatuss, and the like. The computer-readable recording medium further includes transitory media such as data transmission medium. Further, the computer-readable recording medium can be distributed in computer systems connected via a network, wherein the computer-readable codes can be stored and executed in a distributed mode.

Various implementations of the systems and techniques described herein can be realized by a programmable computer. Here, the computer includes a programmable processor, a data storage system (including volatile memory, nonvolatile memory, or any other type of storage system or a combination thereof), and at least one communication interface. For example, the programmable computer may be one of a server, a network apparatus, a set-top box, an embedded apparatus, a computer expansion module, a personal computer, a laptop, a personal data assistant (PDA), a cloud computing system, and a mobile apparatus.

Although exemplary embodiments of the present disclosure have been described for illustrative purposes, those skilled in the art will appreciate that various modifications, additions, and substitutions are possible, without departing from the idea and scope of the claimed invention. Therefore, exemplary embodiments of the present disclosure have been described for the sake of brevity and clarity. The scope of the technical idea of the embodiments of the present disclosure is not limited by the illustrations. Accordingly, one of ordinary skill would understand the scope of the claimed invention is not to be limited by the above explicitly described embodiments but by the claims and equivalents thereof.

What is claimed is:

1. An experienced difficulty analysis method comprising:
acquiring image information including a face region of a learner who learns learning content as biometric information of the learner;
obtaining micro-vibration information of the face region from the biometric information; and
determining experienced difficulty of the learner with respect to the learning content using the micro-vibration information.

2. The experienced difficulty analysis method of claim 1, wherein the image information includes 15 to 60 frames per second.

3. The experienced difficulty analysis method of claim 1, wherein the obtaining of the micro-vibration information includes obtaining the micro-vibration information by extracting position information of the face region from the biometric information.

4. The experienced difficulty analysis method of claim 3, wherein the obtaining of the micro-vibration information includes extracting the position information of the face region from the biometric information by using a pre-learned face classification model, and recognizing a feature point on the face region and/or texture information of the face region by performing face modeling based on the position information of the face region.

5. The experienced difficulty analysis method of claim 4, wherein in the obtaining of the micro-vibration information, the micro-vibration information is obtained by calculating a change amount of the feature point or a change amount of the texture information.

6. The experienced difficulty analysis method of claim 1, wherein the micro-vibration information is micro-vibration information due to vestibular-emotional reflex (VER).

7. The experienced difficulty analysis method of claim 6, wherein the determining of the experienced difficulty includes calculating emotional information and/or physical state information of the learner using the micro-vibration information to determine the experienced difficulty.

8. The experienced difficulty analysis method of claim 7, wherein the determining of the experienced difficulty includes calculating valence and arousal of the learner based on the emotional information and the physical state information, and determining the experienced difficulty based on a combination of the valence and the arousal.

9. The experienced difficulty analysis method of claim 1, further comprising:
obtaining information on a fine skin color change amount in the face region from the biometric information,
wherein in the determining of the experienced difficulty, the information on the fine skin color change amount is further used to determine the experienced difficulty.

10. The experienced difficulty analysis method of claim 9, wherein the determining of the experienced difficulty includes calculating a heart rate of the learner based on the information on the fine skin color change amount, and calculating emotional information and/or physical state information of the learner using the heart rate and the micro-vibration information.

11. The experienced difficulty analysis method of claim 1, further comprising:
updating a method of providing learning content based on the experienced difficulty.

12. The experienced difficulty analysis method of claim 11, further comprising:
acquiring non-biometric information, which is information other than the biometric information, that is collected while the learner performs learning,
wherein the updating of the learning content providing method includes selecting learning content using the non-biological information based on the experienced difficulty.

13. The experienced difficulty analysis method of claim 12, wherein the non-biometric information includes all or part of a time when the learning content is provided, information input by the learner, a time required for the input of the learner, a time when the learner starts to learn the learning content, and whether the learner has used supplemental content.

14. A computer program stored in one or more non-transitory computer-readable recording medium to execute the experienced difficulty analysis method according to claim 1.

15. An experienced difficulty analysis apparatus, comprising:
a biometric information acquisition unit configured to acquire image information including a face region of a learner as biometric information of the learner;
a biometric information analyzer configured to obtain micro-vibration information of the face region from the biometric information; and
an experienced difficulty determination unit configured to determine experienced difficulty of the learner with respect to learning content which the learner learns using the micro-vibration information.

16. The experienced difficulty analysis apparatus of claim 15, further comprising:
an update unit configured to update a method of providing learning content based on the experienced difficulty.
